(19) United States
(12) Patent Application Publication
(10) Pub. No.: US 2025/0099873 A1
(43) Pub. Date: Mar. 27, 2025

(54) PROCESS OF AFFINITY CHROMATOGRAPHY

(71) Applicant: Kashiv BioSciences, LLC, Piscataway, NJ (US)

(72) Inventors: Om Narayan, Ahmedabad (IN); Tarun Kumar Gupta, Ahmedabad (IN); Mayankkumar Thakkar, Ahmedabad (IN)

(21) Appl. No.: 18/972,007

(22) Filed: Dec. 6, 2024

Related U.S. Application Data
(63) Continuation of application No. 17/922,742, filed on Nov. 1, 2022.

(30) Foreign Application Priority Data
May 1, 2020 (IN) 202021018721

(51) Int. Cl. B01D 15/38; B01D 15/42; C07K 16/06; C07K 16/42

(52) U.S. Cl. CPC B01D 15/3809; B01D 15/426; C07K 16/065; C07K 16/4291; C07K 2317/14

(57) ABSTRACT

A process for purification of antibody or fusion protein by affinity chromatography wherein the elution is performed with high salt concentration which reduce turbidity in protein mixture during neutralization steps. The present invention provides an improved process of purifying antibodies through affinity chromatography using high salt-based elution.

### CROSS-REFERENCE TO RELATED APPLICATIONS

This application is a Continuation of U.S. patent application Ser. No. 17/922,742, filed on Nov. 1, 2022, which is a U.S. National Phase Application under 35 U.S.C. §371 of International Patent Application No. PCT/IB2021/053660, filed on May 1, 2021, which claims the benefit of Indian patent application Ser. No. 202021018721, filed on May 1, 2020, the contents of each of which are hereby incorporated by reference in their entirety.

### FIELD OF THE INVENTION

The present invention provides the composition of antibody obtained from affinity chromatography capable to provide low turbidity during the viral inactivation and neutralization. The present invention provides an improved process of purifying antibodies through affinity chromatography using high salt-based elution.

### BACKGROUND OF THE INVENTION

Viruses are potential contaminants in drug manufacturing processes, particularly in cases where biologic drugs are derived from mammalian cell cultures. A source of viral contaminants can be the media used for cell culture or the cell lines producing the biologics of interest.

Monoclonal antibody is widely purified by Protein A affinity chromatography. Affinity chromatography has several advantages since it is an easy, fast, and selective procedure for capturing the target protein. Due to its selectiveness, an affinity-purification step early in the purification chain is commonly introduced. Thereby, the number of successive unit operations can be reduced. The demand for cost-efficient production processes has led to the necessity of optimization of the downstream purification, including the affinity step. Besides the efficient removal of process-related impurities like host cell proteins and DNA, Protein A can be claimed for virus removal. Protein A is eluted at acidic pH, a chemical inactivation of enveloped viruses by denaturation of the envelope proteins is typically performed. Generally, this inactivation step is performed at pH 3.0-4.0. Slower inactivation kinetics are reported at higher pH and lower temperatures.

However, turbid elution pools and high column backpressure are common during elution of monoclonal antibodies (mAbs) by acidic pH in Protein A chromatography, when antibody composition is subjected to viral inactivation treatment.

Filtration is a critical unit operation that is used for primary and secondary clarification during the manufacturing of mammalian cell-based biotherapeutics. However, continuous manufacturing processes require consistent use of filtration over a long period, with potential unpredictable variations in feed stream attributes, which is a challenge currently facing the industry. Moreover, an increase in turbidity will ultimately lead to the use of multiple filters or the use of a filter having a large filtration area, which will directly impact the cost and the operational time of the process. Furthermore, depth filtration is used to remove precipitates and turbidity after neutralization of low pH virus inactivated product. Moreover, positively charged filters can be applied to improve this process step by additional removal of host cell proteins (HCPs) and DNA. Claiming adsorptive depth filters as a virus removal step would enable a further intensification of mAb bioprocessing. The present invention solves this problem by providing a low turbid protein mixture during or post neutralization which requires a small filtration area. Also, to remove precipitates and turbidity after neutralization of low pH virus inactivated product intermediates depth filters are commonly used.

Accordingly, there remains a need in the art to develop the process to remove or reduce turbidity and precipitation during viral inactivation.

### SUMMARY OF THE INVENTION

In an embodiment, the invention provides an improved purification process of antibodies or fragment thereof by using affinity chromatography wherein the elution is performed at a high salt concentration which provides less turbidity or precipitation in eluted composition during virus inactivation & neutralization compared to elution performed at low salt concentration.

In one aspect of this embodiment, the present invention provides high salt concentration during elution is selected from more than 100 mM, more than 110 mM, more than 125 mM, more than 130 mM, more than 140 mM, more than 150 mM, more than 160 mM, more than 170 mM, more than 180 mM, more than 190 mM, more than 195 mM, about 200 mM.

In one aspect of this embodiment, the present invention provides less turbidity or precipitation in eluted composition selected from about 10 NTU, about 20 NTU, about 30 NTU, about 35 NTU, about 36.1 NTU, about 40 NTU, about 42.5 NTU, about 50 NTU, about 60 NTU, about 70 NTU, about 80 NTU, about 90 NTU, about 100 NTU.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration from about 100 mM to about 200 mM with suitable acidic pH;
e. Performing the viral inactivation and neutralization of eluted protein mixture;

Wherein the neutralized protein mixture has turbidity lower than 100 NTU when measured with a standard turbidity meter.

In an embodiment, the elution pH is selected from pH 3 to pH3.5.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having concentration about 125 mM at suitable acidic pH 3.5±0.1;
e. Performing the viral inactivation and neutralization of eluted protein mixture;
   wherein the neutralized protein mixture has turbidity 103 which is lower than the protein mixture eluted with a buffer having concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM at suitable acidic pH 3.5±0.1;
e. Performing the viral inactivation and neutralization of eluted protein mixture;
   wherein the neutralized protein mixture has a turbidity of 36.1 which is lower than the protein mixture eluted with a buffer having a concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM at suitable acidic pH 3.0±0.1;
e. Performing the viral inactivation and neutralization of eluted protein mixture;
   wherein the neutralized protein mixture has a turbidity of 42.5 which is lower than the protein mixture eluted with a buffer having a concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 125 mM;
e. Performing the viral inactivation and neutralization of eluted protein mixture;
   wherein the protein mixture has turbidity lower than 50 NTU when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM;
e. Performing the viral inactivation and neutralization of eluted protein mixture;
   wherein the protein mixture has turbidity lower than 50 NTU when measured with a standard turbidity meter.

In an embodiment, the invention provides a process of purifying a protein mixture comprising;
a. Loading the protein mixture of omalizumab or fragment thereof and impurities onto Affinity column with a suitable buffer;
b. Washing the Affinity column with suitable buffer;
c. Optionally one more wash is performed with a suitable buffer;
d. Eluting the protein mixture enriched with omalizumab with a suitable buffer having a concentration of about 200 mM;
e. Performing the viral inactivation and neutralization of eluted protein mixture;
   wherein the protein mixture has turbidity lower than 20 NTU when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 125 mM;
e. Performing the viral inactivation and neutralization of eluted protein mixture;
   wherein the protein mixture has turbidity lower than 100 NTU when measured with a standard turbidity meter.

### BRIEF DESCRIPTION OF DRAWINGS

FIG. 1: depicts the complete chromatogram.

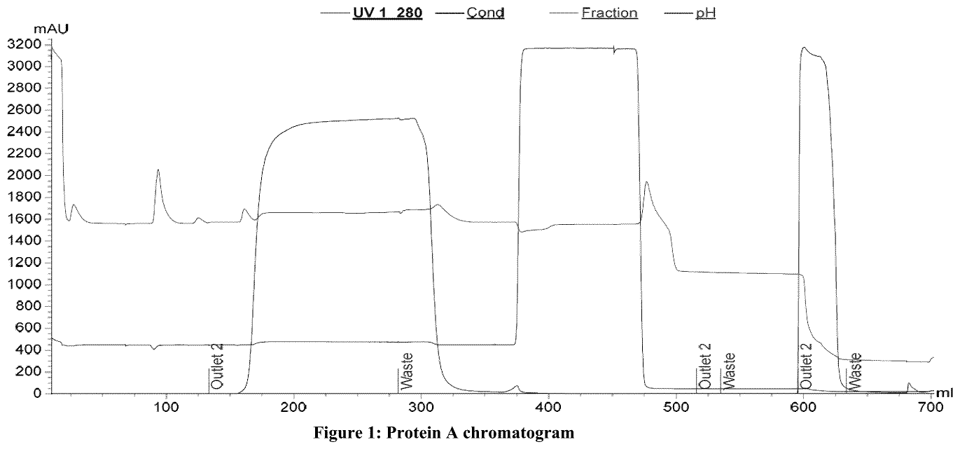

### DETAILED DESCRIPTION OF THE INVENTION

### Definitions

The term “antibody” includes an immunoglobulin molecule comprised of four polypeptide chains, two heavy (H) chains, and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2, and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”). Such decreases in the number and/or activity of viral particles can be on the order of about 1% to about 99%, preferably of about 20% to about 99%, more preferably of about 30% to about 99%, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

As used throughout the specification and in the appended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one of the skill in the art will recognize that, due to the nature of the referenced value, the term “about” can mean more or less than a 10-20% deviation from that value.

Omalizumab (Xolair®) is a recombinant DNA-derived humanized IgG1K monoclonal antibody that selectively binds to human immunoglobulin (IgE). The antibody has a molecular weight of approximately 149 kD. Xolair® is produced by a Chinese hamster ovary cell suspension culture in a nutrient medium containing the antibiotic gentamicin. Gentamicin is not detectable in the final product. Xolair® is a sterile, white, preservative-free, lyophilized powder contained in a single-use vial that is reconstituted with Sterile Water for Injection (SWFI), USP, and administered as a subcutaneous (SC) injection.

Turbidity is the cloudiness or haziness of a fluid caused by large numbers of individual particles that are generally invisible to the naked eye, similar to smoke in the air. The propensity of particles to scatter a light beam focused on them is now considered a more meaningful measure of turbidity in water.

Turbidity measured this way uses an instrument called a nephelometer with the detector set up to the side of the light beam. More light reaches the detector if there are lots of small particles scattering the source beam than if there are few. The units of turbidity from a calibrated nephelometer are called Nephelometric Turbidity Units (NTU).

In protein purification turbidity plays an important role, protein solutions with higher turbidity have higher insoluble particles which may be aggregates or other impurities and have a negative effect on the purity and yield of the protein mixture. High turbidity also damages the cassettes and columns by clogging them. Thus, higher turbidity is an indication of lesser purity and higher impurity concentration and vice versa.

In this process, we are substantially reducing the turbidity of protein A eluate with respect to the protein A input.

In an embodiment, the invention provides an improved purification process of antibodies or fragment thereof by using affinity chromatography wherein the elution is performed at a high salt concentration which provides less turbidity or precipitation in eluted composition compared to elution performed at low salt concentration.

In one aspect of this embodiment, the present invention provides high salt concentration during elution is selected from more than 100 mM, more than 110 mM, more than 125 mM, more than 130 mM, more than 140 mM, more than 150 mM, more than 160 mM, more than 170 mM, more than 180 mM, more than 190 mM, more than 195 mM, about 200 mM.

In one aspect of this embodiment, the present invention provides less turbidity or precipitation in eluted composition selected from about 10 NTU, about 20 NTU, about 30 NTU, about 35 NTU, about 36.1 NTU, about 40 NTU, about 42.5 NTU, about 50 NTU, about 60 NTU, about 70 NTU, about 80 NTU, about 90 NTU, about 100 NTU.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration from about 100 mM to about 200 mM;
e. Performing the viral inactivation and neutralization of eluted protein mixture;
   wherein the protein mixture has turbidity lower than the protein mixture eluted with a buffer having a concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration from about 100 mM to about 200 mM;
e. Performing the viral inactivation and neutralization of eluted protein mixture;
   wherein the protein mixture has turbidity lower than 100 NTU when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having concentration about 125 mM at suitable acidic pH 3.5±0.1;
e. Performing the viral inactivation and neutralization of eluted protein mixture;
   wherein the neutralized protein mixture has turbidity 103 which is lower than the protein mixture eluted with a buffer having concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM at suitable acidic pH 3.5±0.1;
e. Performing the viral inactivation and neutralization of eluted protein mixture;
   wherein the neutralized protein mixture has a turbidity of 36.1 which is lower than the protein mixture eluted with a buffer having a concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM at suitable acidic pH 3.0±0.1;
e. Performing the viral inactivation and neutralization of eluted protein mixture;
   wherein the neutralized protein mixture has a turbidity of 42.5 which is lower than the protein mixture eluted with a buffer having a concentration below 100 mM when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 125 mM;
e. Performing the viral inactivation and neutralization of eluted protein mixture;
   wherein the protein mixture has turbidity lower than 100 NTU when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM;
e. Performing the viral inactivation and neutralization of eluted protein mixture;
   wherein the protein mixture has turbidity lower than 100 NTU when measured with a standard turbidity meter.

In an embodiment, the invention provides a process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM;
e. Performing the viral inactivation and neutralization of eluted protein mixture;
   wherein the protein mixture has turbidity lower than 50 NTU when measured with a standard turbidity meter.

In another embodiment, the use of high salt concentration during protein A elution improves filtration capacity and able to filter the neutralized protein mixture with a small filter area.

In another embodiment, the use of high salt concentration during protein A elution improves filtration capacity by 1 fold or 1.5 fold or 2 fold.

In certain embodiment, the antibody is selected from IgG1, IgG2, IgG3, and IgG4 antibody or fragment thereof and fusion protein. In an embodiment, the IgG1 antibody or fusion protein has isoelectric point from 6 to 9.

The IgG1 antibody or fusion protein are selected from Etanercept, Rituximab, Palivizumab, Infliximab, Trastuzumab, Alemtuzumab, Adalimumab, Ibritumomab, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucirumab, Vedolizumab, Nivolumab, Pembrolizumab, Darucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Sarilumab, Ocrelizumab, Tildrakizumab, Romosozumab, Brolucizumab, Crizanlizumab.

In the preferred embodiment, the antibody is an anti-IgE antibody. In certain embodiment, the anti-IgE antibody is omalizumab.

In an embodiment, the invention provides a process of purifying a protein mixture comprising;
a. Loading the protein mixture of omalizumab or fragment thereof and impurities onto Affinity column with a suitable buffer;
b. Washing the Affinity column with suitable buffer;
c. Optionally one more wash is performed with a suitable buffer;
d. Eluting the protein mixture enriched with omalizumab with a suitable buffer having a concentration of about 200 mM;
e. Performing the viral inactivation and neutralization of eluted protein mixture;
   wherein the protein mixture has turbidity lower than 20 NTU when measured with a standard turbidity meter.

In an embodiment, the invention provides a process of purifying a protein mixture comprising;
a. Loading the protein mixture of omalizumab or fragment thereof and impurities onto Affinity column with a suitable buffer;
b. Washing the Affinity column with suitable buffer;
c. Optionally one more wash is performed with a suitable buffer;
d. Eluting the protein mixture enriched with omalizumab with a suitable buffer having a concentration of about 200 mM;
e. Performing the viral inactivation and neutralization of eluted protein mixture;
   wherein the protein mixture has turbidity lower than 10 NTU when measured with a standard turbidity meter.

In an embodiment, the protein mixture eluted with a buffer having a concentration from 100 mM to 250 mM has turbidity lower than the protein mixture eluted with a buffer having a concentration below 100 mM.

In an embodiment, the protein mixture eluted with a buffer having a concentration from 200 mM has turbidity lower than the protein mixture eluted with a buffer having a concentration of 30 mM.

In an embodiment, the protein mixture eluted with a buffer having a concentration from 100 mM to 125 mM has turbidity lower than the protein mixture eluted with a buffer having a concentration below 30 mM.

In an embodiment, affinity chromatography is selected from Protein A or Protein G. In an embodiment, the affinity chromatography rein is selected from Mabselect, Mabselect SuRe, Mabselect SuRe Lx, Prosep Ultra Plus, Eshmuno A. In the preferred embodiment, the Affinity chromatography resin is Mabselect Sure Lx.

In an embodiment, the equilibration buffer or loading buffer or wash buffer are selected from Sodium Phosphate, Tris-HCl, Tris-Acetate, HEPES, and Glycine-NaOH. In the preferred embodiment, the loading buffer is Tris Acetate.

In certain embodiment, equilibration buffer or loading buffer, or wash buffer is used in combination with salt. In certain embodiment, the salt is selected from sodium Chloride, potassium Chloride, arginine chloride, calcium chloride, and urea. In the preferred embodiment, the salt is Sodium Chloride.

In an embodiment, the equilibration buffer has a concentration range from about 5 mM to about 40 mM. In certain embodiment, the equilibration buffer has a concentration range from about 10 mM to about 25 mM. In the preferred embodiment, the equilibration buffer concentration is about 20 mM.

In certain embodiment, the equilibration buffer or loading buffer or wash buffer optionally comprises a salt selected from about 50 mM to about 400 mM. In an embodiment, the equilibration buffer comprises a salt buffer concentration selected from about 100 mM to about 200 mM. In an embodiment, the equilibration buffer concentration is about 150 mM. In another embodiment, the equilibration buffer concentration is about 100 mM.

In an embodiment, the equilibration buffer or loading buffer, or wash buffer has a conductivity range from about 10 mS/cm to about 20 mS/cm. In an embodiment, the equilibration buffer or loading buffer or wash buffer conductivity is about 15.0 mS/cm to 20.0 mS/cm. In another embodiment, the equilibration buffer or loading buffer, or wash buffer conductivity is about 10.0 mS/cm to 13.0 mS/cm.

In an embodiment, the pH of the equilibration buffer or loading buffer, or wash buffer is selected from about 6.5 to about 7.5. In the preferred embodiment, the Equilibration buffer pH is about 7.0.

In an embodiment, the loading buffer has a concentration range from about 5 mM to about 40 mM. In an embodiment, the loading buffer has a concentration range from about 10 mM to about 30 mM. In the preferred embodiment, the loading buffer concentration is about 20 mM.

In certain embodiment, the affinity chromatography has at least one wash buffer. In another embodiment, the affinity chromatography has three wash buffers.

In an embodiment, the first wash buffer has a concentration range from about 5 mM to about 40 mM. In certain embodiment, the first wash buffer has a concentration range from about 10 mM to about 25 mM. In the preferred embodiment, the first wash buffer concentration is about 20 mM.

In an embodiment, the second wash buffer is selected from sodium phosphate, Tris-HCl, Tris-Acetate, HEPES, and Glycine-NaOH.

In certain embodiment, a second wash buffer is used in combination with salt.

In certain embodiment, the salt is selected from sodium chloride, potassium Chloride, arginine chloride, calcium chloride & urea. In the preferred embodiment, the salt is Sodium Chloride.

In an embodiment, the second wash buffer has a concentration range from about 5 mM to about 40 mM. In certain embodiment, the second wash buffer has a concentration range from about 10 mM to about 25 mM. In the preferred embodiment, the second wash buffer concentration is about 20 mM.

In an embodiment, the second wash buffer has a salt buffer concentration range from about 0.5 M to about 1.5 M. In the preferred embodiment, the second wash buffer concentration is about 1.0 M.

In an embodiment, the second wash buffer has a conductivity range from about 70 mS/cm to about 120 mS/cm. In an embodiment, the second wash buffer has a conductivity range from about 80 mS/cm to about 100 mS/cm. In the preferred embodiment, the second wash buffer conductivity is about 90 mS/cm.

In an embodiment, the pH of the second wash buffer is selected from about 6.5 to about 7.5. In the preferred embodiment, the Second wash buffer pH is about 7.0.

In an embodiment, the second wash buffer further comprises a surfactant which is selected from polysorbate 20, polysorbate 80, triton X-100. In an embodiment, the preferred surfactant is polysorbate 20.

In an embodiment, the percentage of the surfactant in the second wash buffer is from about 0.1% to about 1%.

In an embodiment, the third wash buffer has a concentration range from about 5 mM to about 40 mM. In certain embodiment, the third wash buffer has a concentration range from about 10 mM to about 30 mM. In the preferred embodiment, the third wash buffer concentration is about 20 mM.

In an embodiment, the third wash buffer has a conductivity range from about 0.5 mS/cm to about 2.5 mS/cm. In the preferred embodiment, the third wash buffer conductivity is about 1 mS/cm.

In an embodiment, the pH of the third wash buffer is selected from about 5 to about 6. In the preferred embodiment, the third wash buffer pH is about 5.5.

In an embodiment, the elution buffer is selected from acetic acid, phosphoric acid, HCl. In the preferred embodiment, the elution buffer is acetic acid.

In an embodiment, the elution buffer has a concentration range selected from about 100 mM to about 250 mM. In the preferred embodiment, the elution buffer has a concentration range of about 200 mM.

In an embodiment, the elution buffer has a conductivity range from about 0.2 mS/cm to about 0.7 mS/cm. In an embodiment, the elution buffer has a conductivity range from about 0.5 mS/cm to about 0.6 mS/cm. In an embodiment, the elution buffer has a conductivity range from about 0.2 mS/cm to about 0.3 mS/cm.

In an embodiment, the pH of the elution buffer is selected from 2.5 to about 3.5. In the preferred embodiment, the elution buffer pH is about 3.0.

In certain embodiment, elution is performed in linear gradient. In certain embodiment, elution is performed in step gradient.

In an embodiment, where the elution peak collection starts at an ascending value of about 2.5 AU/cm and ends at a descending value of about 2.5 AU/cm.

In an embodiment, where the elution peak collection starts at an ascending value of about 0.25 AU/cm and ends at a descending value of about 0.25 AU/cm.

In an embodiment, the invention provides the antibody composition having turbidity selected from less than about 100 NTU, less than about 50 NTU, less than about 30 NTU, less than about 10 NTU obtained from affinity chromatography wherein the elution buffer has a concentration of about 200 mM.

In another embodiment, the invention provides a purification process of antibodies or fragments thereof by using affinity chromatography wherein the elution is performed at low salt concentration.

In another embodiment, the invention provides a purification process of antibodies or fragment thereof by using affinity chromatography wherein the elution is performed at low salt concentration, which does not reduce the turbidity compared to elution performed with a high salt concentration of the eluted protein mixture during viral inactivation.

In an embodiment, the equilibration is performed for about 3 CV's to about 10 CV's. In a preferred embodiment, the equilibration is performed for about 5 CV's. In an embodiment, the equilibration is performed until the equilibration buffer conductivity endpoint is achieved.

In an embodiment, the amount of protein loaded onto the column during loading is at a range of about 10 g/l to about 40 g/l. In an embodiment, the amount of protein loaded onto the column during loading is at a range from about 10 g/l to about 50 g/l.

In an embodiment, the first wash is performed for at least 1 to about 5 CV's. In the preferred embodiment, the first wash is performed for 3 CV's. In an embodiment, the first wash is performed until the buffer conductivity endpoint is achieved.

In an embodiment, the second wash is performed for at least 1 CV to about 5 CV's. In the preferred embodiment, the second wash is performed for 5 CV's. In an embodiment, the second wash is performed until the buffer conductivity endpoint is achieved.

In an embodiment, the third wash is performed for at least 4 CV's to about 8 CV's. In the preferred embodiment, the third wash is for 7 CV's. In an embodiment, the third wash is performed until the buffer conductivity endpoint is achieved.

In an embodiment, the residence time of the protein in the column during protein A purification has a range from about 2 minutes to about 6 minutes. In the preferred embodiment, the residence time of the protein in the column is about 4 minutes.

### EXAMPLE 1

### Purification of Monoclonal Antibody Performed by Protein A Chromatography (Affinity) with 25 mM Acetate Buffer, pH 3.5

All chromatographic processes were carried out using an AKTA Pure 150 system from Cytiva. The concentration of protein samples was determined by measuring absorbance at 280 nm using Shimadzu Spectrophotometer. Mabselect Sure LX resin media obtained from Cytiva. Vantage columns were obtained from Millipore Corporation. Turbidity measurements were measured using TN100 Portable Turbidimeter from Thermo scientific. All Chemicals were obtained from JTB or Merck Millipore and were of GMP grade.

A monoclonal antibody molecule capable to bind to IgE molecule expressed in Chinese Hamster Ovary (CHO) cell line is captured using Protein A (Mab Select Sure LX, Cytiva) packed in Vantage columns VL11×30 Millipore column. The residence time is 4 min for all the phases. After equilibration with Tris Acetate+150 mM NaCl, pH 6.8-7.2 clarified harvest is loaded at ≤40 mg/mL of the resin. After loading column washes with equilibration buffer followed by wash 2 and wash 3 buffers followed by elution with 25 mM Acetate, pH 3.5±0.1 buffer as mentioned in Table 1. The affinity chromatography step is operated in bind and eluate mode and collection is done from 500 mAU (2.5 AU/cm) ascending to 500 mAU (2.5 AU/cm) descending of the peak. Elute is further subjected to viral inactivation and neutralization. Turbidity was measured at protein A elute stage.

For virus inactivation pH of protein, A Elute is adjusted to pH 3.5 with 1N HCl and incubates at room temperature for 50 minutes. After 50 minutes of incubation, a sample is neutralized to pH 6.2with a 1M tris base for 20 min. NTU of neutralized protein A elute is measured with a turbidity meter. Neutralized protein A elute is filtered with a 0.2 μm filter. The experimental design for the Protein A step and NTU data are summarized in Table 1 and Table 5 respectively.

TABLE 1
Experimental design for Protein A chromatography

| | | Residence | Column |
|---|---|---|---|
| | | Time | Volume |
| Step | Buffer | (min) | (CV) |
| WFI | WFI | 4 | 2-3 |
| Sanitization | 0.1M NaOH | 4 | 2-3 |
| Equilibration | 20 mM Tris-acetate + 150 mM | 4 | 3-5 |
| | NaCl, pH 7.0 ± 0.2 | | |
| Load | Clarified Harvest | 4 | Till end |
| | | | of load |
| | | | volume |
| Wash 1 | 20 mM Tris-acetate + 150 mM | 4 | 3-5 |
| | NaCl, pH 7.0 ± 0.2 | | |
| Wash 2 | 20 mM Tris-acetate + 1M NaCl + | 4 | 3-5 |
| | 0.1% Polysorbate 20, pH 7.0 ± 0.2 | | |
| Wash 3 | 30 mM Tris-acetate, pH 5.5 ± 0.2 | 4 | 5-7 |
| Elution | 25 mM Acetate, pH 3.5 ± 0.1 | 4 | 3 |
| Neutralization | 20 mM Tris-acetate + 150 mM | 4 | 2-3 |
| wash | NaCl, pH 7.0 ± 0.2 | | |
| Sanitization | 0.1M NaOH | 4 | 2-3 |
| Neutralization | 20 mM Tris-acetate + 150 mM | 4 | 3-5 |
| wash | NaCl, pH 7.0 ± 0.2 | | |
| Storage | 2% Benzyl alcohol in Wash 3 | 4 | 2-3 |
| | buffer | | |

### EXAMPLE 2

### Purification of Monoclonal Antibody Performed by Protein A Chromatography (Affinity) with 125 mM Acetate Buffer, pH 3.5

All chromatographic processes were carried out using an AKTA Pure 150 system from Cytiva. The concentration of protein samples was determined by measuring absorbance at 280 nm using Shimadzu Spectrophotometer. Mabselect Sure LX resin media obtained from Cytiva. XK50/40 column was obtained from Cytiva. Turbidity measurements were measured using TN100 Portable Turbidimeter from Thermo scientific. All Chemicals were obtained from JTB or Merck Millipore and were of GMP grade.

A monoclonal antibody molecule capable to bind to IgE molecule expressed in the Chinese Hamster Ovary (CHO) cell line is captured using Protein A (Mab Select Sure LX, Cytiva) packed in the XK50/40 column. The residence time is 4 min for all the phases. After equilibration with Tris Acetate+150 mM NaCl, pH 6.8-7.2 clarified harvest is loaded at ≤40 mg/mL of the resin. After loading column washes with equilibration buffer followed by wash 2 and wash 3 buffers followed by elution with 125 mM Acetate, pH 3.5±0.1 buffer as mentioned in Table 2. The affinity chromatography step is operated in bind and eluate mode and collection is done from 500 mAU (2.5 AU/cm) ascending to 500 mAU (2.5 AU/cm) descending of the peak. Elute is further subjected to viral inactivation and neutralization. Turbidity was measured at protein A elute stage.

For virus inactivation pH of Protein A Elute is adjusted to pH 3.5 with 1N HCl and incubates at room temperature for 50 minutes. After 50 minutes of incubation, a sample is neutralized to pH 6.2 with a 1M tris base for 20 min. NTU of neutralized protein A elute is measured with a turbidity meter. Neutralized protein A elute is filtered with a 0.2 μm filter. The experimental design for the Protein A step and NTU data are summarized in Table 2 and Table 5 respectively.

TABLE 2
Experimental design for Protein A chromatography

| | | Residence | Column |
|---|---|---|---|
| | | Time | Volume |
| Step | Buffer | (min) | (CV) |
| WFI | WFI | 4 | 2-3 |
| Sanitization | 0.1M NaOH | 4 | 2-3 |
| Equilibration | 20 mM Tris-acetate + 150 mM | 4 | 3-5 |
| | NaCl, pH 7.0 ± 0.2 | | |
| Load | Clarified Harvest | 4 | Till end |
| | | | of load |
| | | | volume |
| Wash 1 | 20 mM Tris-acetate + 150 mM | 4 | 3-5 |
| | NaCl, pH 7.0 ± 0.2 | | |
| Wash 2 | 20 mM Tris-acetate + 1M NaCl + | 4 | 3-5 |
| | 0.1% | | |
| | Polysorbate 20, pH 7.0 ± 0.2 | | |
| Wash 3 | 30 mM Tris-acetate, pH 5.5 ± 0.2 | 4 | 5-7 |
| Elution | 125 mM Acetate, pH 3.5 ± 0.1 | 4 | 3 |
| Neutralization | 20 mM Tris-acetate + 150 mM | 4 | 2-3 |
| wash | NaCl, pH 7.0 ± 0.2 | | |
| Sanitization | 0.1M NaOH | 4 | 2-3 |
| Neutralization | 20 mM Tris-acetate + 150 mM | 4 | 3-5 |
| wash | NaCl, pH 7.0 ± 0.2 | | |
| Storage | 2% Benzyl alcohol in Wash 3 | 4 | 2-3 |
| | buffer | | |

### EXAMPLE 3

### Purification of Monoclonal Antibody Performed by Protein A Chromatography (Affinity) with 200 mM Acetate Buffer, pH 3.5

All chromatographic processes were carried out using an AKTA Pure 150 system from Cytiva. The concentration of protein samples was determined by measuring absorbance at 280 nm using Shimadzu Spectrophotometer. Mabselect Sure LX resin media obtained from Cytiva. Vantage columns were obtained from Millipore Corporation. Turbidity measurements were measured using TN100 Portable Turbidimeter from Thermo scientific. All Chemicals were obtained from JTB or Merck Millipore and were of GMP grade.

A monoclonal antibody molecule capable to bind to IgE molecule expressed in Chinese Hamster Ovary (CHO) cell line is captured using Protein A (Mab Select Sure LX, Cytiva) packed in Vantage columns VL11×30 Millipore column. The residence time is 4 min for all the phases. After equilibration with Tris Acetate+150 mM NaCl, pH 6.8-7.2 clarified harvest is loaded at ≤40 mg/mL of the resin. After loading column washes with equilibration buffer followed by wash 2 and wash 3 buffers followed by elution with 200 mM Acetate, pH 3.5±0.1 buffer as mentioned in Table 1. The affinity chromatography step is operated in bind and eluate mode and collection is done from 500 mAU (2.5 AU/cm) ascending to 500 mAU (2.5 AU/cm) descending of the peak. Elute is further subjected to viral inactivation and neutralization. Turbidity was measured at protein A elute stage.

For virus inactivation pH of protein, A Elute is adjusted to pH 3.5 with 1N HCl and incubates at room temperature for 50 minutes. After 50 minutes of incubation, a sample is neutralized to pH 6.2 with a 1M tris base for 20 min. NTU of neutralized protein A elute is measured with a turbidity meter. Neutralized protein A elute is filtered with a 0.2 μm filter. The experimental design for Protein A step and NTU data are summarized in Table 3 and Table 5 respectively.

TABLE 3
Experimental design for Protein A chromatography

| | | Residence | Column |
|---|---|---|---|
| | | Time | Volume |
| Step | Buffer | (min) | (CV) |
| WFI | WFI | 4 | 2-3 |
| Sanitization | 0.1M NaOH | 4 | 2-3 |
| Equilibration | 20 mM Tris-acetate + 150 mM | 4 | 5 |
| | NaCl, pH 7.0 ± 0.2 | | |
| Load | Clarified Harvest | 4 | Till |
| | | | loading |
| | | | volume |
| Wash 1 | 20 mM Tris-acetate + 150 mM | 4 | 3-5 |
| | NaCl, pH 7.0 ± 0.2 | | |
| Wash 2 | 20 mM Tris-acetate + 1M NaCl + | 4 | 3-5 |
| | 0.1% PS20, pH 7.0 ± 0.2 | | |
| Wash 3 | 30 mM Tris-acetate, pH 5.5 ± 0.2 | 4 | 5-7 |
| Elution | 200 mM Acetate, pH 3.5 ± 0.1 | 4 | 3 |
| Neutralization | 20 mM Tris-acetate + 150 mM | 4 | 2-3 |
| wash | NaCl, pH 7.0 ± 0.2 | | |
| Sanitization | 0.1M NaOH | 4 | 2-3 |
| Neutralization | 20 mM Tris-acetate + 150 mM | 4 | 3-5 |
| wash | NaCl, pH 7.0 ± 0.2 | | |
| Storage | 2% Benzyl alcohol in Wash 3 | 4 | 2-3 |
| | buffer | | |

### EXAMPLE 4

### Purification of Monoclonal Antibody Performed by Protein A Chromatography (Affinity) with 200 mM Acetate Buffer, pH 3.0

All chromatographic processes were carried out using an AKTA Pure 150 system from Cytiva. The concentration of protein samples was determined by measuring absorbance at 280 nm using Shimadzu Spectrophotometer. Mabselect Sure LX resin media obtained from Cytiva. Vantage column was obtained from Millipore Corporation. Turbidity measurements were measured using TN100 Portable Turbidimeter from Thermo scientific. All Chemicals were obtained from JTB or Merck Millipore and were of GMP grade.

A monoclonal antibody molecule capable to bind to IgE molecule expressed in Chinese Hamster Ovary (CHO) cell line is captured using Protein A (Mab Select Sure LX, Cytiva) packed in Vantage columns VL11×30 column. The residence time is 4 min for all the phases. After equilibration with Tris Acetate+100 mM NaCl, pH 6.8-7.2 clarified harvest is loaded at ≤40 mg/mL of the resin. After loading column washes with equilibration buffer followed by wash 2 and wash 3 buffers followed by elution with 200 mM Acetate, pH 3.0±0.1 buffer as mentioned in Table 3. The affinity chromatography step is operated in bind and eluate mode and collection is done from 50 mAU (0.25 AU/cm) ascending to 50 mAU (0.25 AU/cm) descending of the peak. Elute is further subjected to viral inactivation a neutralization. Turbidity was measured at protein A elute stage.

For virus inactivation pH of protein, A Elute is adjusted to pH 3.5 with 1N Acetic acid and incubates at room temperature for 50 minutes. After 50 minutes of incubation, a sample is neutralized to pH 6.2 with 1M Tris base. NTU of neutralized protein A elute is measured with a turbidity meter. Neutralized protein A elute is filtered with a 0.2 μm filter. The experimental design for the Protein A step and NTU data are summarized in Table 4 and Table 5 respectively.

TABLE 4
Experimental design for Protein A

| | | Residence | Column |
|---|---|---|---|
| | | Time | Volume |
| Step | Buffer | (min) | (CV) |
| WFI | WFI | 4 | 2-3 |
| Sanitization | 0.1M NaOH | 4 | 2-3 |
| Equilibration | 20 mM Tris-acetate + 100 mM | 4 | 5 |
| | NaCl, pH 7.0 ± 0.2 | | |
| Load | Clarified Harvest | 4 | Till |
| | | | loading |
| | | | volume |
| Wash 1 | 20 mM Tris-acetate + 100 mM | 4 | 3 |
| | NaCl, pH 7.0 ± 0.2 | | |
| Wash 2 | 20 mM Tris-acetate + 1M NaCl, | 4 | 5 |
| | pH 7.0 ± 0.2 | | |
| Wash 3 | 20 mM Tris-acetate, pH 5.5 ± 0.2 | 4 | 5-7 |
| Elution | 200 mM Acetate, pH 3.0 ± 0.1 | 4 | 3 |
| Neutralization | 20 mM Tris-acetate + 100 mM | 4 | 2-3 |
| wash | NaCl, pH 7.0 ± 0.2 | | |
| Sanitization | 0.1M NaOH | 4 | 2-3 |
| Neutralization | 20 mM Tris-acetate + 100 mM | 4 | 3-5 |
| wash | NaCl, pH 7.0 ± 0.2 | | |
| Storage | 2% Benzyl alcohol in Wash 3 | 4 | 2-3 |
| | buffer | | |

TABLE 5
Turbidity data of Protein A Chromatography step

| | | | Neutralized |
|---|---|---|---|
| | | Protein A | Protein A |
| Example | Elution Buffer | Elute (NTU) | Elute (NTU) |
| Example 1 | 25 mM Acetate, pH 3.5 ± 0.1 | 3.3 | 227.0 |
| Example 2 | 125 mM Acetate, pH 3.5 ± 0.1 | 10.2 | 103.0 |
| Example 3 | 200 mM Acetate, pH 3.5 ± 0.1 | 6.7 | 36.1 |
| Example 4 | 200 mM Acetate, pH 3.0 ± 0.1 | 5.4 | 42.5 |

In conclusion, the salt concentration of elution buffer and pH during Protein A chromatography elution drastically affect the turbidity in neutralized samples or protein mixture.

As shown in Example 1 when protein get eluted with 25 mM acetate buffer, pH 3.5 shows turbidity 227 NTU which is significantly higher than protein eluted with 125 mM and 200 mM acetate buffer, pH 3.5, 227.0 NTU and 103.0 NTU were observed respectively in neutralized protein A elute. When compares to 200 mM Acetate, pH 3.5±0.1 and 200 mM Acetate, pH 3.5±0.1 buffer, 36.1 NTU, and 42.5 NTU were observed in neutralized protein A elute. Lower NTU value directly improves the subsequent depth filtration performance as well as decreases the area requires for the unit operation.

We claim:

1. A process of purifying a protein mixture comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration from about 100 mM to about 200 mM at suitable acidic pH;
e. Performing the viral inactivation and neutralization of eluted protein mixture;
wherein the neutralized protein mixture has turbidity lower than the protein mixture eluted with a buffer having a concentration below 100 mM when measured with a standard turbidity meter.

2. The process according to claim 1, wherein the high salt concentration during elution is selected from more than 100 mM, more than 110 mM, more than 125 mM, more than 130 mM, more than 140 mM, more than 150 mM, more than 160 mM, more than 170 mM, more than 180 mM, more than 190 mM, more than 195 mM, about 200 mM.

3. The process according to claim 1, wherein the suitable buffer concentration during elution step (d) is selected from about 100 mM to about 200 mM.

4. The process according to claim 1, wherein the suitable buffer concentration during elution step (d) is about 125 mM.

5. The process according to claim 4, wherein the elution buffer provides the turbidity in neutralized protein mixture below about 103 NTU.

6. The process according to claim 1, wherein the suitable buffer concentration during elution step (d) is about 200 mM.

7. The process according to claim 6, wherein the elution buffer provides the turbidity in neutralized protein mixture selected from 60 NTU, 50 NTU, 42.5 NTU, 40 NTU, 36.1 NTU, 35 NTU, 30 NTU.

8. The process according to claim 7, wherein the turbidity in neutralized protein mixture is 36 NTU.

9. The process according to claim 7, wherein the turbidity in neutralized protein mixture is 42.5 NTU.

10. The process according to claim 1, wherein the suitable acidic pH is selected from about pH 3 to about pH 3.5.

11. The process of purifying a protein mixture according to claim 1, comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having concentration about 125 mM at suitable acidic pH 3.5±0.1;
e. Performing the viral inactivation and neutralization of eluted protein mixture;
wherein the neutralized protein mixture has turbidity 103 which is lower than the protein mixture eluted with a buffer having concentration below 100 mM when measured with a standard turbidity meter.

12. The process of purifying a protein mixture according to claim 1, comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM at suitable acidic pH 3.5±0.1;
e. Performing the viral inactivation and neutralization of eluted protein mixture;
wherein the neutralized protein mixture has a turbidity of 36.1 which is lower than the protein mixture eluted with a buffer having a concentration below 100 mM when measured with a standard turbidity meter.

13. The process of purifying a protein mixture according to claim 1, comprising;
a. Loading the protein mixture of antibody or fragment thereof and impurities onto Affinity column with a suitable buffer;
b. Washing the Affinity column with suitable buffer;
c. Optionally one more wash is performed with a suitable buffer;
d. Eluting the protein mixture enriched with an antibody of interest with a suitable buffer having a concentration of about 200 mM at suitable acidic pH 3.0±0.1;
e. Performing the viral inactivation of eluted protein mixture;
wherein the neutralized protein mixture has a turbidity of 42.5 which is lower than the protein mixture eluted with a buffer having a concentration below 100 mM when measured with a standard turbidity meter.

14. The process according to claim 1, wherein antibody is selected from IgG1, IgG2, IgG3, and IgG4 antibody or fragment thereof and fusion protein.

15. The process according to claim 41, wherein the IgG1 antibody or fusion protein has isoelectric point from 6 to 9.

16. The process according to claim 41, wherein the IgG1 antibody or fusion protein are selected from Etanercept, Rituximab, Palivizumab, Infliximab, Trastuzumab, Alemtuzumab, Adalimumab, Ibritumomab, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucirumab, Vedolizumab, Nivolumab, Pembrolizumab, Darucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Sarilumab, Ocrelizumab, Tildrakizumab, Romosozumab, Brolucizumab, Crizanlizumab.

17. The process according to claim 1, wherein antibody is an anti-IgE antibody.

18. The process according to claim 17, wherein anti-IgE antibody is omalizumab.

19. A process of purifying a protein mixture according to claim 1, comprising;
a. Loading the protein mixture of omalizumab or fragment thereof and impurities onto Affinity column with a suitable buffer;
b. Washing the Affinity column with suitable buffer;
c. Optionally one more wash is performed with a suitable buffer;
d. Eluting the protein mixture enriched with omalizumab with a suitable buffer having a concentration of about 200 mM;
e. Performing the viral inactivation and neutralization of eluted protein mixture;
Wherein the neutralized protein mixture has turbidity lower than 20 NTU when measured with a standard turbidity meter.

20. The process according to claim 1, wherein the protein mixture eluted with a buffer having a concentration from 200 mM has turbidity lower than the protein mixture eluted with a buffer having a concentration of 30 mM.

21. The process according to claim 1 wherein the protein mixture eluted with a buffer having a concentration from 100 mM to 125 mM has turbidity lower than the protein mixture eluted with a buffer having a concentration below 30 mM.

22. The process according to claim 1, wherein the affinity chromatography is selected from Protein A or Protein G.

23. The process according to claim 1, wherein the elution buffer is selected from acetic acid, Phosphoric acid, and HCl.

24. The process according to claim 1, wherein the elution buffer is acetic acid.

25. The process according to claim 1, the pH of the elution buffer is selected from 2.5 to about 3.5.

26. The process according to claim 1, the elution buffer pH is about 3.0.
